(19) United States
(12) Patent Application Publication
(10) Pub. No.: US 2005/0232979 A1
(43) Pub. Date: Oct. 20, 2005

(54) Multi-layer collagenic article useful for wounds healing

(75) Inventor: Shmuel Shoshan, Moza-Illit (IL)

Correspondence Address: LOWE HAUPTMAN GILMAN AND BERNER, LLP, 1700 DIAGONAL ROAD, SUITE 300 /310, ALEXANDRIA, VA, 22314, US

(73) Assignee: YISSUM RESEARCH DEVELOPMENT COMPANY OF THE HEBREW UNIVERSITY OF JERUSALEM, JERUSALEM (IL)

(21) Appl. No.: 10/517,048

(22) PCT Filed: Jun. 3, 2002
(86) PCT No.: PCT/IL02/00430

§ 371 Date: Dec. 3, 2004

(51) Int. Cl. A61L 15/00

(52) U.S. Cl. 424/445

(57) ABSTRACT

The invention relates to a multi-layer collagen article useful for wound healing, comprising at least two layers; wherein at least one layer, facing the wound side, is comprising an effective amount of non or partially cross-linked collagen; and at least one layer comprising an effective amount of highly cross-linked collagen matrices. The invention also relates to a method for the production of collagen article.

### FIELD OF THE INVENTION

The present invention relates to collagenic article useful for wound healing. More specifically, the invention relates to a multi-layer collagen article useful for wound healing, comprising at least two layers; wherein at least one layer, facing the wound side, is comprising an effective amount of non or partially cross-linked collagen; and at least one layer comprising an effective amount of highly cross-linked collagen matrices. The present invention further relates to the method for the production of said collagenic article for wound healing.

### BACKGROUND OF THE INVENTION

Repair of injured tissue is a sequence of events in which cells with distinct functions are attracted to the wound, proliferate and secrete extracellular matrix materials to restore structure and function. Activation of platelets and blood coagulation are first in the sequence of events, followed by the appearance of polymorphonuclear leaukocytes, monocytes and lymphocytes at the site of the injury. Fibroblasts, or fibroblasts-like cells, which appear next, are of particular interest since it is these cells which produce most of the extracellular connective tissue matrix, and are thus responsible for proper repair process. Mediators originating from platelets, monocytes, macrophages, lymphocytes, and connective tissue themselves regulate migration to the site of injury, proliferation and metabolic activity of fibroblasts. Adequate repair is associated with a time and concentration dependent exposure of fibroblasts to these mediators. Migration of fibroblasts to the wound occurs by a process called chemotaxis, i.e., by a directional migration of cells against a concentration gradient of a chemo-attractant substance. Attractants for fibroblasts belong to different molecular species including collagen, the principal extracellular structural protein of the animal body, and to a variety of growth factors, all believed to be involved in the tissue repair process.

At least twenty types of mammalian collagen have been isolated, mainly in bones, skin, cartilages and around nerves and blood vessels. The common characteristic amongst them is a three-stranded helix, consisting of three polypeptide chains, called alpha-chains. All alpha-chains have the same configuration, but differ in the composition and sequence of their amino acids. Type I collagen is composed of two alpha₁-chains and one alpha₂-chain and is the principal extracellular material of skin, tendon and bone. In this patent, “collagen” will be defined as mainly native Type I collagen, namely consisting the triple domain of the molecule. In addition, all collagen chains contain regions at each end, which are not helical. These regions are thought to be responsible for the immunogenicity associated with most collagen preparations, and this property can, in large part, be mitigated by removal of these regions to produce “atelopeptide” collagen. The removal can be accomplished by digestion with proteolytic enzymes such as trypsin or pepsin. These non-helical telopeptide regions are however, required to form most of the cross-links, which are responsible for stability of the fibrillar structure in native collagen, since they contain aldehydes capable of cross-linkage; atelopeptide collagen must be cross-linked artificially if it is desired to obtain this characteristic.

Natural collagen fibers are basically water insoluble in mature tissues because of covalent intermolecular cross-links that convert collagen into an infinite crosslinked network. Dispersal and solubilization of native collagen can be achieved by treatment with various proteolytic enzymes which disrupt the intermolecular bonds and removes immunogenic non-helical end regions without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). Subsequent purification of the solubilized collagen can be accomplished by repeated precipitation at high pH or ionic strength, washing and resolubilization. Introduction of covalent cross-links into the purified soluble collagen is an important aspect in stabilizing and restructuring the material for biomedical use.

Collagen also attains an important role in several regulatory functions relevant to the amount and the quality of the extracellular matrix and the scar tissue in the healing wound. Thus, it has been further established that the rate of collagen synthesis is regulated in the presence of collagen pro-peptides, whereas the chemotactic properties are regulated by a concentration gradient formed by peptides originating from the metabolic breakdown process initiated by collagenase, which attacks more readily non-cross linked collagen molecules. Furthermore, it has been shown recently that non-crossed linked collagen enhances the expression of collagen type I mRNA and hence facilitates the closure of dermal wounds (Redlich, M. et al., Matrix Biology 17:667-71 (1998)). Following this approach, a dental dressing was prepared, where soluble collagen and cross-linked collagen were mixed, and their mixture was cross-linked by a cross-linking agent (See Japan Patent No. 3,294,209) in order to reduce the solubility of the non-crosslinked collagen. Nevertheless, incorporating active soluble collagen with cross-linked collagen in one dressing but in separated integrated layers to yield a healing “all-collagen” wound dressing has not published.

Various synthetic materials, e.g., cyanoacrylates and other polymers, have been proposed to render collagen more suitable as biomedical adhesives. (See Shimizu et al., Biomat. Med. Dev. Art. Org., 6(4): 375-391 (1978); and Buonocore, M., Adhesion in Biological Systems, R. S. Manly, ed., Academic Press, New York, 1970, Chap. 15). In many instances, the prior modified collagen-based adhesives suffer from various deficiencies which include (1) crosslinking/polymerization reactions that generate exothermic heat, (2) long reaction times, and (3) reactions that are inoperative in the presence of oxygen and physiological pH ranges, (4) many of the prior modified collagen-based adhesives contain toxic materials, hence rendering it unsuitable for biomedical use (see, for example, U.S. Pat. No. 3,453,222). Still another disadvantage of solid cross-linked collagen implants are (4) the requirement for surgical implantation by means of incision, (5) lack of deformability and flexibility. There are hence no safe, efficacious adhesives for medical use with soft tissue.

Said disadvantages of synthetic adhesives has led the development of biologically derived adhesives, such as fibrin based glues, as bonding materials. Nevertheless, commercial fibrin tissue adhesives are derived from human plasma and hence pose potential health risks such as adverse immunogenic reactions and transmission of infectious agents, e.g., Hepatitis B virus. Moreover, the bond strength imparted by such adhesives are relatively weak compared to collagen adhesives (see De Toledo, A. R. et al. Asso. for Res. in Vision and Ophthalmology, Annual Meeting Abstract, Vol. 31, 317 (1990)).

Collagen has been used previously as a structural ingredient, providing the desired three-dimensional matrix of pharmaceutical one-layer sponges or of thin membrane sheets (See U.S. Pat. Nos. 3,157,524; 3,514,518; 3,628,974; 3,939,831; 4,320,201; 4,374,121; 4,409,322; 4,412,947; 4,418,601; 4,600,533; 4,655,980; 4,689,399; 4,703,108; 4,971,954; 4,837,285; 4,937,323; 5,73,376; PCT Patent Applications WO 86/03122 and WO 90/00060, and European Patent Applications 167828; 187014).

Bi-layer sponges, composed of collagen and other polymers were used to entrapped various drugs in the layer facing the wound (See U.S. Pat. No. 4,642,118; Japan Pat. No. 4364120A2). Similarly, collagenic wound dressings composed three-layered structure were issued, such as in the arrangement of (i) an adhesive, (ii) a cross-linked collagen matrix, and (iii) a multi-layer polymer film (See U.S. Pat. Nos. 4,841,962; 4,950,699, and British Patent 1,347,582). It is thus indicated that there is no technology to produce a preparation that would satisfy the need of both non-crosslinked and highly crosslinked collagen in one dressing, thus providing both cell-growth promoting effect and protection for injured tissue

### SUMMARY OF THE INVENTION

In accordance with the present invention, a multi-layer collagen article useful for wound healing, comprising at least two layers; wherein at least one layer, facing the wound side, is comprising an effective amount of non or partially cross-linked collagen; and at least one layer comprising an effective amount of highly cross-linked collagen matrices is described. Further object of the invention is said multi-layer wound healing dressing comprising at least one sponge collagen matrix or at least one thin membranal collagen sheet. Still another object of the invention is wherein said collagen wound healing dressing is comprising one or more drug species, biological or synthetic elastomers, biological glues, pH buffers, plasticizers, stabilizing agents and drying enhancers. Another embodiment of the present invention is a method for the production of collagen aforementioned article, comprising but not limited to the operations of preparing non-crosslinked collagens; non-enzymatic glycosylating said matrices; integrating the layers by means of thermally reconstituting said formed collagen fibers by monosaccharide-aldehyde; washing and lyophilizing said formed crossed-linked layer, and dressing a wound, wherein the smooth surface of the collagen non or partially crossed-linked collagen layer is facing the surface of said wound. Another preferred embodiment of the present invention is a method for enhancing wound healing, by means of administrating said multi-layer collagen, as previously defined wherein said collagen wound healing dressing onto wounds, cuts or burns in dermal or oral cavities injuries.

### DETAILED DESCRIPTION OF THE INVENTION

In accordance of the present invention, both the collagen molecule and its fibers must be stabilized by intramolecular and intermolecular covalent cross-links in order to function as a structural protein, which is firstly aimed to restore to health the wounded tissue, and secondly to provide the protection to the subsequently formed scar tissue.

It is well established that non-enzymatic glycosylation of collagen in vitro as well as in vivo by covalent attachment of the carbonyl group of a saccharine (i.e., via Millard Reaction) to a free amino group of peptide bound lysine and hydroxylysine and the subsequent condensation and formation of Schiff-base followed by the rearrangement into more stable Amadori products. Thus, the interaction of a reducing sugar with non-crosslinked collagen fibers in vitro may bring about interfibrillar stable cross-links and consequent decreased solubility. This non-enzymatic collagen glycation is normal biological process and has no adverse effect on the tissue.

The present invention provides a method to obtain a preparation made of a metabolically very active layer of non-crosslinked collagen facing the wound bed and an integrated non-enzymatically cross-linked and biologically compatible layer of collagen on top of it. This endows the wound dressing with both enhanced healing capacity and a protective quality over the wound bed during the repair process. Such a dressing also serves a vehicle for delivery of a variety of substances, which may be needed for specific situation in order to enhanced healing.

According to the embodiment of the present invention, an aqueous sterile solution of non-crosslinked native collagen in phosphate buffer (ionic strength 0.4; pH 7.6) is made at a concentration of 2.0 to 3.0 mg ml⁻¹. The solution is heated at 37° C. for 6 to 24 hours or less, until native collagen fibers are reconstituted. Then, a solution of a monosaccharide-aldehyde, such as glyceraldehydes, at a concentration of 0.1 M to 0.5M in the same buffer is overlaid over the gel to cover it with a 1 mm to 3 mm layer and left at 37° C. for about 6 hours. In this patent, soluble collagen is defined as a collagen that has an average molecular weight of less than 400,000, preferably having a molecular weight of about 300,000. This particular soluble collagen is also advantageous because it is the atelopeptide form of the collagen.

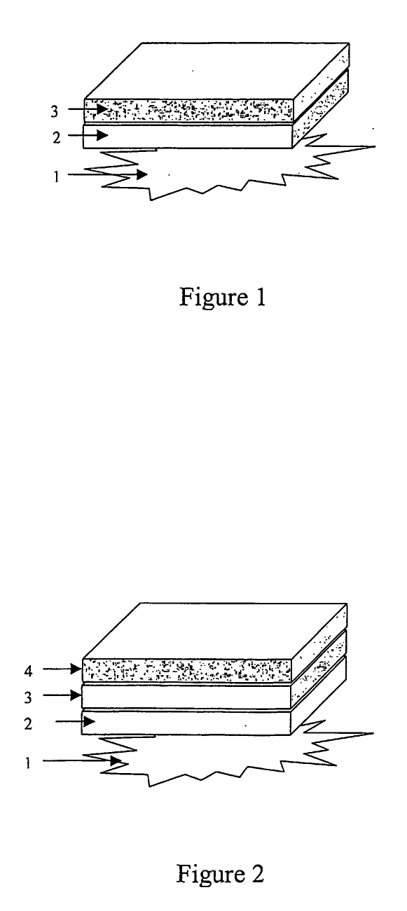

In one preferred embodiment of the patent, a superficial layer of reconstituted water immiscible, highly cross-linked collagen fibers (FIG. 1, #3), completely integrated with the previously made non-crosslinked collagen layer (FIG. 1, #2), is thus formed. Following this, the gel is thoroughly washed with distilled water by carefully pouring it over the gel to remove the phosphate and the carbohydrate. Then, the collagenic article is lyophilized to provide a multi-layered sponge to be used as a dressing or implant for wounds of any kind (FIG. 1, #1). The upper surface of the sponge containing the non-crosslinked collagen will be dressed onto the wound.

To optimized desirable characteristics of a preferred collagen multi-layered sponge and to meet specific needs of a particular wound, it is possible to enrich the dressing with a variety of substances according to the specific requirements of a given wound, e.g., angiogenic factors in case of ischemic wounds or antibacterial agents in case of infected wounds etc.

To optimize desirable characteristics of a preferred collagen-containing sponge, it is possible to add to the collagen-based composition various additives. Such desirable characteristics include flexibility, stability, accelerated drying time and a pH compatible with the active ingredient to be utilized.

To improve flexibility, a suitable plasticizer can be used. Suitable plasticizers include polyethylene glycol and glycerol, preferably glycerol. Such plasticizers can be present in an amount from zero to about 100% of the weight of collagen present, preferably from about 10 to about 30% of the weight of collagen present, most preferably about 20% of the weight of collagen present. To improve the stability of the active ingredient, a suitable stabilizing agent can be used in the collagen. Suitable stabilizing agents include most sugars, preferably mannitol, lactose, and glucose, more preferably mannitol. Such stabilizing agents can be present in an amount from zero to about 5% of the weight of collagen present, preferably about 1% of the weight of collagen present.

According to another preferred embodiment, a sheet article according to the invention is arranged in a multi-layer sheet (FIG. 2), whereas the side of the inner non-crosslinked collagen of the wound dressing (#2) is facing the wound surface (#1), the highly cross-linked collagen outer side (#3) is on top of the sheet, and partially cross-linked collagen (#4), in one or more layers, in one or more extent of cross-linking, are sandwiched between the inner and outer layers.

### EXAMPLE

Two differently prepared non-crosslinked collagens were used for subsequent non-enzymatic cross-linking:

i. From dermis of guinea pigs made lathyritic by the lathyrogen beta-amino-propionitrile. The lathyrogen administered i.p. at a dose of 1 mg per 1 gbw daily for 15 days. Other nitriles, such as aminoacetonitrile may also be used. The animals where then killed with an overdose of pentothal and the non-crosslinked collagen was extracted from the dermis with cold 0.15 N NaCl, and purified by a TCA-ethanol procedure, according to Gross (J. Exp. Med. 107, 1247,1958).
ii. Non-crosslinked collagen was also obtained by feeding guinea pigs with penicillamine, 10 mg per 1 gdw, for 21 days. The non-crosslinked collagen was then extracted and treated as that from the lathyritic animals.
   The purified collagen samples were freeze-dried by lyophilization, and before use, solutions of 3 mg ml⁻¹ were prepared in phosphate buffer, pH 7.6 and ionic strength 0.45. These solutions were than subject to non-enzymatic glycosylation by incubating them with an aqueous 0.2 M glyceraldehyde solution at ambient temperature for 72 hours. The cross-linked collagen fibers were then precipitated with cold water, collected and freeze-dried by lyophilization. A sample of the lyophilized collagen was immediately put in the original volume of cold 0.5 M acetic acid to let it dissolve by gentle shaking in the cold room for 24 hours. The other lyophilized samples were kept for different time periods till 20 days. To determine their solubility, the samples at each time point were centrifuged and both the insoluble precipitate and the solubilized collagens in the supernatant were determined by their hydroxyproline content. Solubility was expressed in the supernatant as percent from the total.

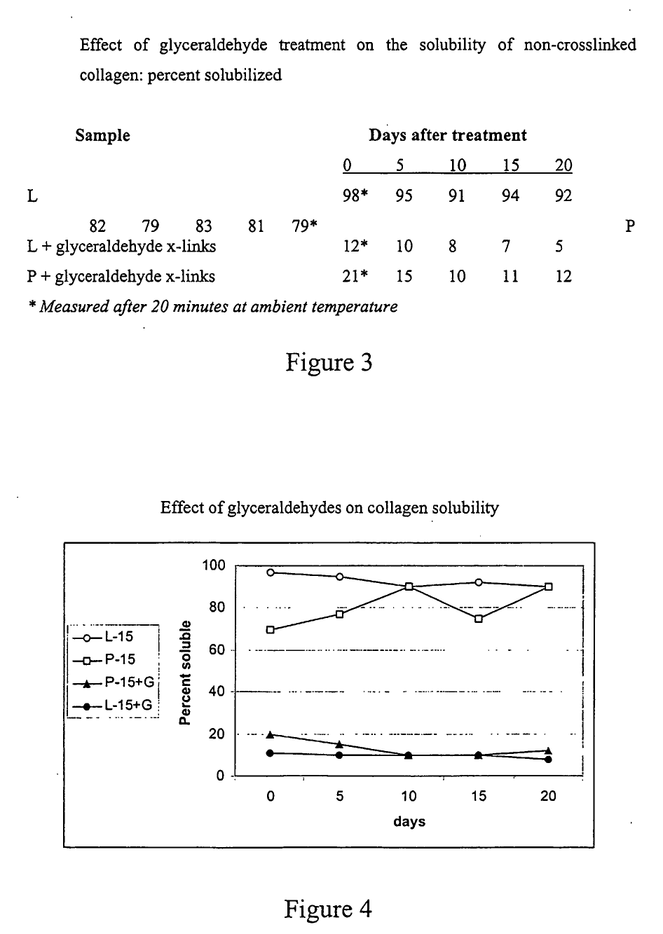

The results are shown in FIG. 3 and in FIG. 4, where L indicates collagen from lathyritic and P denotes collagen from penicillamine treated animals, both non-crosslinked. The drastic decrease in solubility is indicative of highly crosslinked collagen. The solubility of normally crosslinked collagen, such as obtained from normal animals with acid extraction ranges between 35% and 40%.

A bi-layer collagen sponge was prepared according to the following steps:

1. 3 ml of non-crosslinked collagen solution in phosphate buffer, pH 7.6 and ionic strength 0.4 is poured into 10 ml beaker and allowed to thermally reconstitute the collagen fibers at 37° C. for 6 hours.
2. A 0.2 M glyceraldhyde solution in the same buffer is overloaded over the collagen fibers to form a 0.5 mm to 10 mm layer. This is kept at ambient temperature for 72 hours, All the glyceraldehyde is thereby covalently bound to the amino groups of the lysines and hydroxylysines of the non-crosslinked collagen thus forming a highly crosslinked collagen layer of about 0.5 mm to 10 mm on top of the non-crosslinked layer beneath.
3. The gel is then washed with several changes of distilled water and made into a sponge by lyophilization.
4. The sponge is removed from the beaker.
5. For dressing a wound, the smooth surface of the sponge, which had been at the flat bottom of the beaker, will be facing the surface of the wound.

To test the in vivo effect of the collagen layers, 36 full thickness dermal excision wounds were inflicted on the back of 18 guinea pigs, 2 wounds each, under general anesthesia using a punch biopsy of 6 mm. The animal experiments had been carried out in accordance with the permission of the Institutional Committee for Laboratory Animal Care. Twelve wounds were left as untreated controls. Twelve wounds were dressed with a normally cross-linked collagen sponge, and twelve wounds were dressed with the collagen multi-layer. One half of the animals were killed after 5 days and the second half after 10 days. The results were assessed by measuring the wound closure, by using a microscopic grid, following the preparation of histological sections. Closure was expressed as percent advance of epithelium relative to initial wound width.

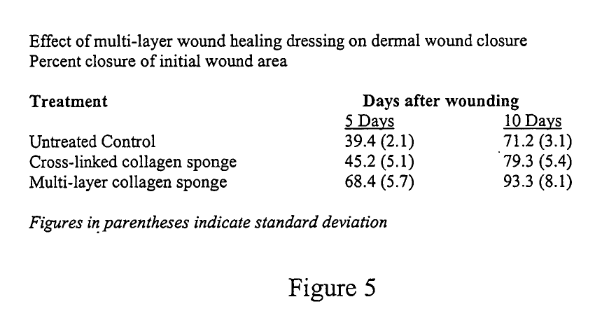

The results are shown in FIG. 5, which clearly demonstrate the advantage of the multi-layer wound healing dressing for enhancing the healing of a full thick dermal excision wound.

1. A multi-layer collagen article useful for wound healing, comprising at least two layers; wherein at least one layer, facing the wound side, is comprising an effective amount of non or partially cross-linked collagen; and at least one layer comprising an effective amount of highly cross-linked collagen matrices.

2. A multi-layer collagen article useful for wound healing according to claim 1, wherein said multi-layer wound healing dressing comprising at least one sponge collagen matrix.

3. A multi-layer collagen article useful for wound healing according to claim 1, wherein said a multi-layer wound healing dressing comprising at least one thin membranal collagen sheet.

4. A method for the production of collagen article, as defined in claim 1, comprising but not limited to the operations of preparing non-crosslinked collagens; non-enzymatic glycosylating said matrices; integrating the layers by means of thermally reconstituting said formed collagen fibers by monosaccharide-aldehyde; washing andlyophilizing said formed crossed-linked layer, and dressing a wound, wherein the smooth surface of the collagen non or partially crossed-linked collagen layer is facing the surface of said wound.

5. A method of enhancing wound healing, by means of administrating a multi-layer collagen, as defined in claim 1.

6. The method according to claim 5, wherein said collagen wound healing dressing administrated onto wounds, cuts or burns in dermal or dental injured tissues.
